(19) United States
(12) Patent Application Publication
(10) Pub. No.: US 2024/0354808 A1
(43) Pub. Date: Oct. 24, 2024

(54) ADAPTIVE SENSORY REINFORCEMENT GENERATION IN DISPENSING ENVIRONMENTS

(71) Applicant: Wayne Fueling Systems LLC, Austin, TX (US)

(72) Inventor: Scott R. Negley, III, Austin, TX (US)

(21) Appl. No.: 18/638,364

(22) Filed: Apr. 17, 2024

Related U.S. Application Data
(60) Provisional application No. 63/497,501, filed on Apr. 21, 2023.

(51) Int. Cl. G06Q 30/0251

(52) U.S. Cl. CPC G06Q 30/0268

(57) ABSTRACT

A method for adaptively determining and generating a sensory reinforcement in a dispensing environment is provided. The method can include receiving first data characterizing a state of a dispensing environment and second data characterizing a graphical communication to be provided on a display screen within the dispensing environment. The method can also include determining a sensory reinforcement to be provided in the dispensing environment based on the first data and/or the second data. The method can further include generating the sensory reinforcement and providing the sensory reinforcement and/or the graphical communication within the dispensing environment. Related systems, devices, and computer-readable mediums are also provided.

### CROSS-REFERENCE TO RELATED APPLICATIONS

This application claims priority to U.S. Provisional Patent Application No. 63/497,501 filed Apr. 21, 2023, entitled “Adaptive Sensory Reinforcement Generation In Dispensing Environments” which is hereby incorporated herein by reference in its entirety.

### FIELD

Methods and systems for adaptively determining and generating a sensory reinforcement in a dispensing environment are provided.

### BACKGROUND

Dispensing environments can provide fuel, electricity, gas, or similar consumable products or energy-generating materials to vehicles via dispensers. Vehicle operators can enter the dispensing environment to purchase fuel, gas, or electricity for their vehicle. Often, dispensing environments offer other items or services for purchase, such as food, drinks, vehicle maintenance equipment, vacuum, air, repair services, or car washes.

Marketing efforts by dispensing environment operators can be limited to traditional modes of marketing such as word of mouth, signage, flyers, posters, social media, or advertising within the dispensing environment to attract customers and command revenue/sales sufficient to sustain profitability. However, at times it can be difficult to reach potential customers via these traditional marketing channels. Influencing potential customers closer to the point of sale within the dispensing environment can lead to higher rates of purchasing, increased sales, and greater customer loyalty.

Existing dispensers can be configured with displays to provide marketing outreach materials, such as video and audio advertisements. However, the provision of these advertisements may be unrelated to a potential customer present within the dispensing environment or may be randomly displayed on a particular schedule or maybe displayed as a continuous loop that is unrelated to a particular schedule or any particular product or service available within the dispensing environment.

Aroma or scents can be an effective mechanism to influence a potential customer to purchase an item or service that corresponds to, smells like, or causes a potential customer to think of the item or service. There exists a lack of dispensing solutions which can coincidently and adaptively provide aroma or scents with displayed marketing or advertising materials for an item or service to increase the likelihood of a customer purchasing the item or service associated with the aroma or scent.

### SUMMARY

Methods and systems for adaptively determining and generating sensory reinforcements, such as aromas and fragrances, in a dispensing environment are provided. Related apparatus, techniques, and articles are also described.

In one aspect, a method is provided. In an embodiment, the method can include receiving first data characterizing a state of a dispensing environment. The method can further include receiving second data characterizing a graphical communication to be provided on a display screen within the dispensing environment. The method can also include determining a sensory reinforcement to be provided in the dispensing environment based on the first data and/or the second data. The method can further include generating the sensory reinforcement and providing the sensory reinforcement and/or the graphical communication within the dispensing environment.

In some embodiments, the state of the dispensing environment can include at least one of a presence of at least one patron within the dispensing environment, a time of day, a local ambient condition of the dispensing environment is located, a flow of vehicle traffic within the dispensing environment, or a utilization of equipment or services within the dispensing environment. In some embodiments, the sensory reinforcement can include at least one of an aroma, a smell, or a fragrance. In some embodiments, the sensory reinforcement can be generated on a predetermined schedule. In some embodiments, the sensory reinforcement can be provided concurrently with the graphical communication.

In some embodiments, the sensory reinforcement can be provided non-concurrently with the graphical communication. In some embodiments, the sensory reinforcement can be a mitigating scent configured to remedy undesirable odors present within the dispensing environment. In some embodiments, the second data characterizing the graphical communication can be determined based on at least one attribute of a vehicle present within the dispensing environment and an identity of an operator of the vehicle. In some embodiments, determining the sensory reinforcement can further include determining at least one attribute of the sensory reinforcement, the at least one attribute including a type, a medium, a strength, a concentration, a composition, or an emission duration of the sensory reinforcement. In some embodiments, the sensory reinforcement can be determined based on a predictive model trained in a machine learning process to output a predicted sensory reinforcement based on the graphical communication or the at least one attribute of the sensory reinforcement.

In another aspect, a dispenser is provided. In an embodiment, the dispenser can include a display, an olfactory generator, at least one sensor, at least one data processor, and a memory storing instructions, which when executed by the at least one data processor cause the at least one data processor to perform operations including receiving first data characterizing a state of a dispensing environment including the dispenser, the state based on sensor data received from the at least one sensor. The operations can also include receiving second data characterizing a graphical communication to be provided on the display of the dispenser. The operations can further include determining a sensory reinforcement to be provided via the olfactory generator of the dispenser based on the first data and/or the second data. The operations can also include generating, by the olfactory generator of the dispenser, a sensory reinforcement and providing the sensory reinforcement via the olfactory generator of the dispenser and/or providing the graphical communication via the display of the dispenser.

In some embodiments, the state of the dispensing environment can include at least one of a presence of at least one patron within the dispensing environment, a time of day, a local ambient condition of the dispensing environment is located, a flow of vehicle traffic within the dispensing environment, or a utilization of equipment or services within the dispensing environment. In some embodiments, the sensory reinforcement can include at least one of an aroma, a smell, or a fragrance. In some embodiments, the sensory reinforcement can be generated on a predetermined schedule. In some embodiments, the sensory reinforcement can be provided concurrently with the graphical communication.

In some embodiments, the sensory reinforcement can be provided non-concurrently with the graphical communication. In some embodiments, the sensory reinforcement can be a mitigating scent configured to remedy undesirable odors present within the dispensing environment. In some embodiments, the second data characterizing the graphical communication can be determined based on at least one attribute of a vehicle present within the dispensing environment and an identity of an operator of the vehicle. In some embodiments, determining the sensory reinforcement can further include determining at least one attribute of the sensory reinforcement, the at least one attribute including a type, a medium, a strength, a concentration, a composition, or an emission duration of the sensory reinforcement. In some embodiments, the sensory reinforcement can be determined based on a predictive model trained in a machine learning process to output a predicted sensory reinforcement based on the graphical communication or the at least one attribute of the sensory reinforcement.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### BRIEF DESCRIPTION OF DRAWINGS

The embodiments described above will be more fully understood from the following detailed description taken in conjunction with the accompanying drawings. The drawings are not intended to be drawn to scale. For purposes of clarity, not every component may be labeled in every drawing. In the drawings:

FIG. 1 is a process flow diagram illustrating an example method of adaptively determining, generating, and providing a sensor reinforcement within a dispensing environment.

FIG. 2 is a process flow diagram illustrating an example method of adaptively determining, generating, and providing sensory reinforcements and graphical communications within a dispensing environment;

FIG. 3 is a diagram illustrating an exemplary embodiment of a dataflow executed by an example embodiment of a system for adaptively determining, generating, and providing sensory reinforcements and graphical communications performing the methods of FIGS. 1 and 2;

FIG. 4 is a diagram illustrating an exemplary embodiment of a system for adaptively determining, generating, and providing sensory reinforcements and graphical communications within a dispensing environment according to the methods of FIGS. 1 and 2;

FIG. 5A is a side perspective view of one embodiment of a dispenser configured to adaptively generate sensory reinforcements and graphical communications;

FIG. 5B is a front perspective view of the dispenser shown in FIG. 5A;

FIG. 6 is a system block diagram illustrating one embodiment of a dispensing system;

FIG. 7 is a system block diagram of a dispenser in the dispensing system of FIG. 6;

FIG. 8 is a diagram illustrating a perspective view of an embodiment of the dispenser of FIGS. 6 and 7 configured to dispense a liquid product;

FIG. 9 is a side perspective view of an embodiment of a dispenser configured to dispense electricity; and

FIG. 10 is a front perspective view of an embodiment of a dispenser configured to dispense a gaseous product.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

As described earlier, operators of dispensing environments seek to maximize sales and profits using existing resources in the most efficient manner possible. Traditional advertising and promotional marketing materials may fall short of capturing existing or new customers in order to increase sales and profits. Many traditional advertising and promotional materials and delivery methods provide generic materials to all users of the dispenser environment. As a result, users may be turned off by the promotional offers and fail to participate in the promotional offer. Thus, the probability a user will accept, agree, or otherwise participate or utilize a promotional offer may be low. Advertising and promotions within dispensing environments can be less effective to generate sales if the dispensing environment is unpleasant, dirty, or has bad odor, which may cause potential customers to ignore marketing campaigns and disregard advertisements or promotions. In a worst case, a customer may leave a dispensing environment all together if they feel the dispensing environment is unclean or has unpleasant odors.

An improved promotional system would be able to adaptively determine and provide promotional offers based on specific characteristics of a user of the dispensing environment or the user's vehicle. For example, determining a user is a frequent visitor to a dispensing environment to purchase fuel, electricity, or gas, the system could determine that additional promotional offers could be provided to the user, such as a free car wash, or discounted product or service offering. The system, based on identifying the user, could adaptively determine and provide a graphical communication corresponding to a promotional offer that is specifically tailed for the identified user and/or their vehicle. Adaptively determining and providing promotional offers can increase sales and profits for dispensing environment operators where generic promotional and marketing techniques fall short to engage new and existing customers in a purchase. The likelihood or probability of the user accepting the promotional offer to make an unplanned or additional purchase can be used to determine a value of the promotion so that the user is presented with a promotion they are most likely to accept. The graphical communications can be displayed or provided to the user in a variety of manners described herein. In addition, sensory reinforcement of promotional offers can be provided by an improved promotional system in the form of aroma, scent, or fragrance generated to correspond to product or service available within the dispensing environment or a particular promotional offer and the graphical communication identifying such displayed on a dispenser or other point of sale device within the dispensing environment.

In general, a system for adaptively determining, generating, and providing graphical communications and associated aromas, fragrances, or scents in a dispensing environment, and methods for use thereof, are provided. The graphical communications can correspond to advertisements, promotions, coupons, rebates, or marketing materials associated with product or service offerings available in the dispensing environment or in related locations or contexts associated with an operator of the dispensing environment. The graphical communication could correspond to an offer for a fuel discount, an electricity discount, a gas discount, a car wash, a beverage, a diagnostic or repair service, or the like. The system can dynamically and adaptively determine promotional offers of varying values that can be presented to a user such that the user is most likely to accept the offer and the operator of the dispensing environment is most likely to accrue a sale or purchase that they may not have achieved in the absence of the graphical communication. The system can determine and adaptively provide sensory reinforcement associated with a product or service identified by the graphical communication. The sensory reinforcement can include an aroma, scent, or fragrance that can be generated to correspond with a product or service, as well as with the generated graphical communication providing the promotional offer associated with a product or service available within the dispensing environment.

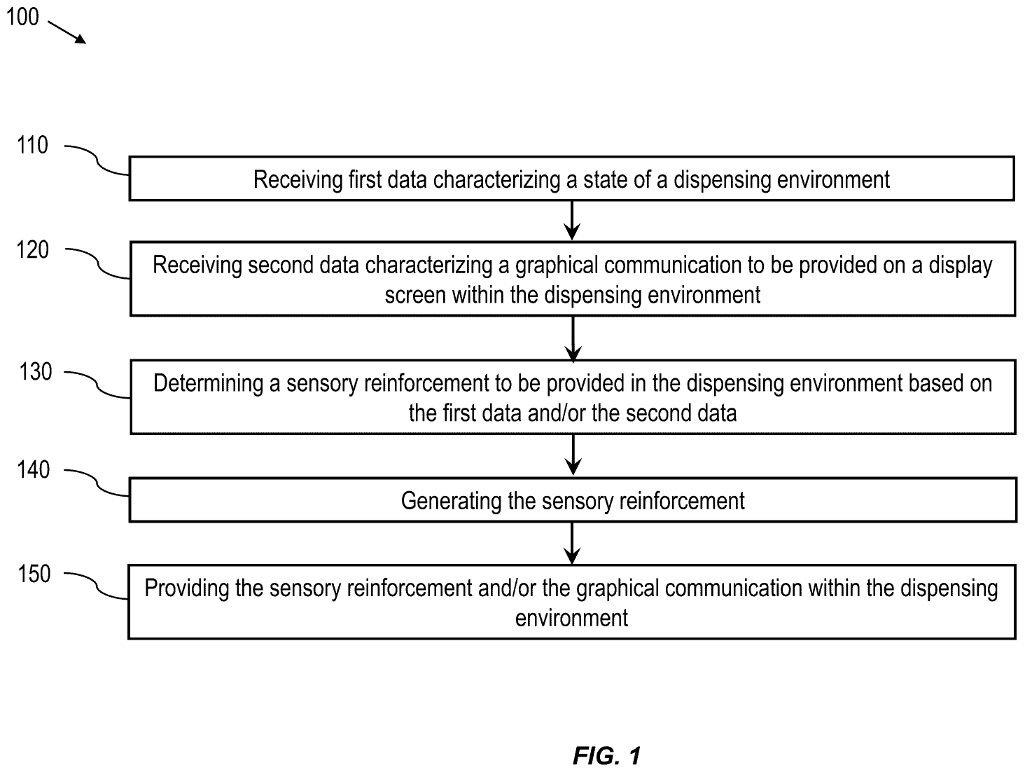

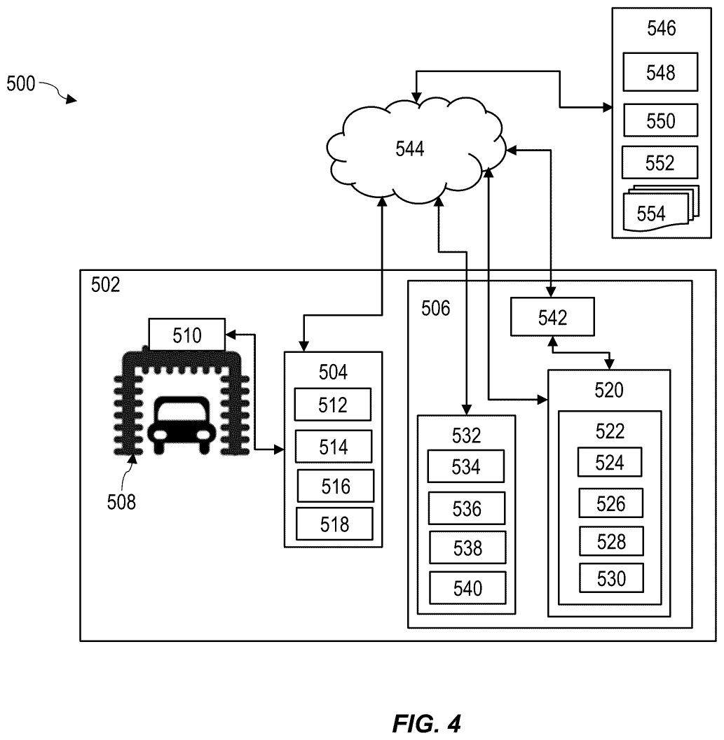

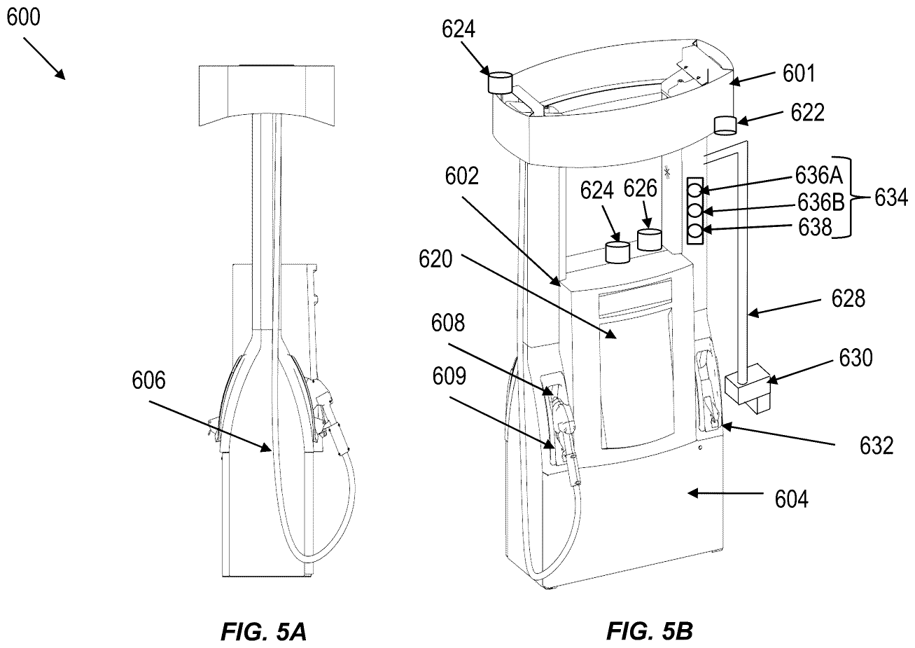

FIG. 1 is a process flow diagram illustrating an example method 100 of adaptively determining, generating, and providing a sensory reinforcement within a dispensing environment. At 110, first data characterizing a state of a dispensing environment is received. The state can be determined based on first data received from one or more sensors 626 configured within the dispensing environment as described in relation to FIGS. 4 and 5A-5B. The state can indicate the presence of patrons, or vehicles. In some embodiments, the state can indicate a time of day, a local ambient condition (e.g., weather) present at the dispensing environment, a rate, or a flow of traffic within the dispensing environment, or a utilization of equipment or services available within the dispensing environment, such as utilization of a car wash or a utilization of a coffee machine or a food vending machine.

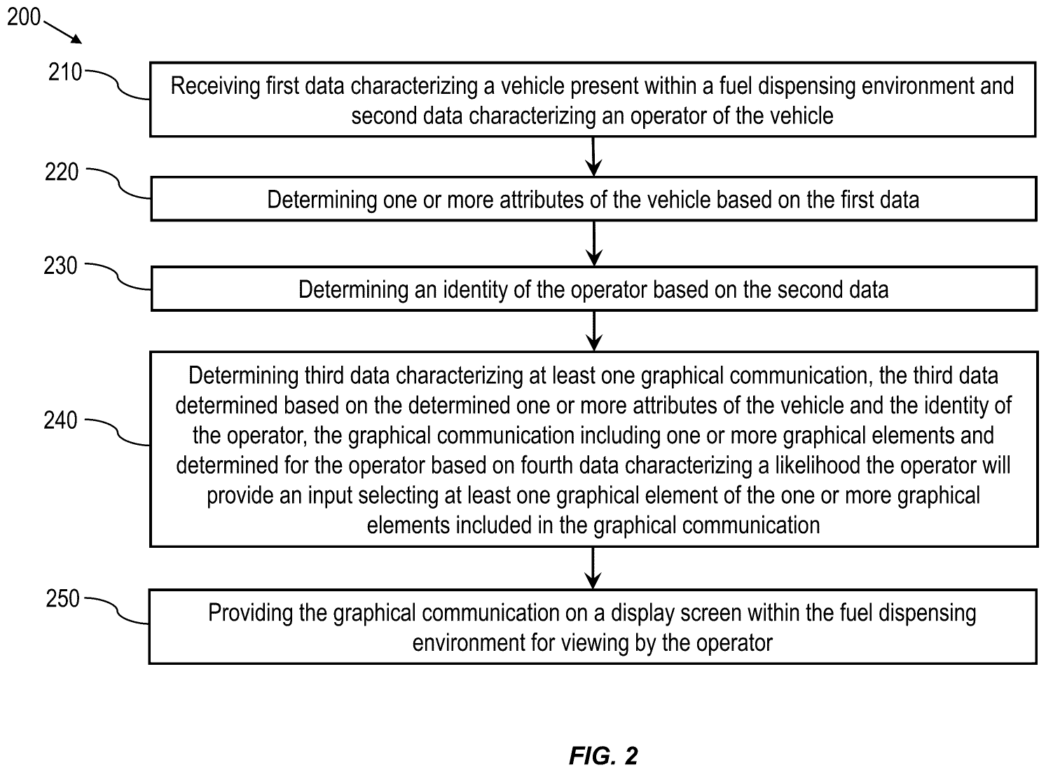

At 120, second data characterizing a graphical communication to be provided on a display screen 528 or 620 within the dispensing environment can be received. The second data can correspond to the third data characterizing a graphical communication determined at step 240 of method 200 described in relation to FIG. 2.

At 130, a sensory reinforcement to be provided in the dispensing environment can be determined based on the first data and/or the second data. In some embodiments, the sensory reinforcement can be determined and provided in the absence of the first data or the second data. For example, the sensory reinforcement can be generated based on a predetermined schedule.

At 140, the sensory reinforcement can be generated. For example, the OG 634 can receive control signals from the control module 612 to cause the scent generators 636 to generate a sensory reinforcement, such as an aroma, a smell, or a fragrance.

At 150, the scent generators 636 can provide the sensory reinforcement and/or the graphical communication within the dispensing environment. In some embodiments, the sensory reinforcement can be provided concurrently with the graphical communication display on the dispenser 600. In other embodiments, the sensory reinforcement can be provided non-concurrently with a graphical communication, such as when it is determined that undesirable odors are present from fuel or exhaust fumes. In this embodiment, the scent generators 636 can control to generate a mitigating smell, scent, or aroma to remedy the undesirable odors within the fuel dispensing environment.

FIG. 2 is a process flow diagram illustrating an example process 200 of some implementations of the current subject matter that can provide for adaptive determination and provision of graphical communications in a dispensing environment.

At 210, a first data characterizing a vehicle present within a dispensing environment can be received. The first data can include image data, such as still image data or video image data, which includes the vehicle within the fuel dispensing environment. Additionally, at 210, second data characterizing an operator of the vehicle can also be received. The second data can include image data, such as still image data or video image data, which includes the operator of the vehicle. In some embodiments, the second data can include data characterizing the operator of the vehicle that is received prior to the operator's presence within the dispensing environment. In some embodiments, the vehicle can display a loyalty program identifier, such as a sticker or decal, on the vehicle that can be included in the image data. The loyalty program identifier can identify the vehicle and the operator of the vehicle.

In some embodiments, the first data and the second data can include non-image data. For example, the first data and the second data can include loyalty program data identifying the vehicle and the operator of the vehicle. The loyalty program data can be associated with the dispensing environment. In some embodiments, the loyalty program data can include a loyalty program account number, a credit card associated with the operator of the vehicle and the loyalty program account, a loyalty program status, and a purchase history of transactions performed by the operator of the vehicle. In some embodiments, the first data and the second data can be received prior to the vehicle entering the dispensing environment. For example, the first data and the second data can be received as a result of the operator of the vehicle logging into a loyalty program application configured on a computing device associated with the operator of the vehicle.

At 220, one or more attributes of the vehicle can be determined based on the first data. The first data can include one or more attributes of the vehicle such as a make, model, size, shape, style, fuel-type, height, length, width, color, window style, type, age, or condition of the vehicle.

The one or more attributes of the vehicle can be determined from the first data using a computer vision algorithm or the like configured to receive the first data as an input and to output attributes of the vehicle. In some embodiments, the computer vision algorithm can include an image classifier. The image classifier can perform pixel-wise classification and segmentation analysis of the first data to determine the attributes of the vehicle. In some embodiments, the image classifier can be implemented using one or more image classification methods, including template matching, low-order Markov chains, Hilbert space-filling curves, Markov meshes, or dependency trees.

In some embodiments, the one or more attributes of the vehicle can be determined using loyalty program data stored in a database. For example, upon receiving first data associated with a loyalty program of the dispensing environment, the one or more attributes of the vehicle can be determined based on the loyalty program data.

At 230, an identity of the operator of the vehicle can be determined based on the second data. The identity of the operator can be determined from the second data using a computer vision algorithm configured to receive the second data as an input and to output the identity of the vehicle operator. In some embodiments, the computer vision algorithm can include an image classifier. The image classifier can perform pixel-wise classification and segmentation analysis of the second data to determine the attributes of the vehicle. In some embodiments, the image classifier can be implemented using one or more image classification methods, including template matching, low-order Markov chains, Hilbert space-filling curves, Markov meshes, or dependency trees. In some embodiments, the identity of the operator can include a sex of the vehicle operator.

In some embodiments, the identity of the operator can be determined using loyalty program data stored in a database. For example, upon receiving second data associated with a loyalty program of the dispensing environment, the identity of the vehicle operator can be determined based on the loyalty program data.

At 240, third data characterizing at least one graphical communication can be determined. The third data can be determined based on the one or more attributes of the vehicle and/or the identity of the vehicle operator. In some embodiments, the third data can include a graphical communication associated with products or goods offered for sale within the dispensing environment. In other embodiments, the third data can include a graphical communications associated with good or services offered for sale outside of the dispensing environment, such as at a neighboring business location or a preferred partner/vendor location away from the location of the dispensing environment.

The graphical communication can be associated with a promotion, advertisement, marketing offer, or the like for goods or services available within the dispensing environment. The graphical communication can identify the promotion being offered and can include graphical elements corresponding to a price, a location, a timing, a service, and/or a product or item associated with the promotion. The graphical elements can also include user-input selections configured to indicate the user has accepted or declined a promotion identified in the graphical communication. The graphical communication and/or the graphical elements can include textual data, graphical data, and/or computer-executable content, which when executed can cause a computing system to take further action. For example, upon receiving an input to a graphical element accepting a free car wash promotion, the graphical element can be configured to execute programmatic functionality to cause a voucher for the free car wash to be printed at a dispenser or a point-of-sale device within the dispensing environment, or transmitted to a computing device of the vehicle operator.

In some embodiments, the third data can be generated based on a particular schedule, a calendar, seasonally, or the like and may not be associated with data that is associated with a vehicle (e.g., the first data) or an operator of the vehicle (e.g., the second data). For example, the third data can be determined based on a day/night cycle or schedule identifying periods of daylight and periods of darkness. A graphical communication associated with a promotion (or the availability) of coffee could be provided during early daylight hours (e.g., 6 AM-10 AM) but would not be provided during nighttime hours. Similarly, a graphical communication associated with food or alcohol could be provided during evening hours, but may not be provided during the early daylight hours.

In some embodiments, the third data can be generated based on state information associated with the dispensing environment, such as the presence or absence of vehicles, a measure of cleanliness of the dispensing environment, or an amount (or rate) of usage of the dispensing environment.

At 250, the graphical communication can be provided on a display screen within the dispensing environment for viewing by the operator. For example, graphical communications can be displayed to the vehicle operator at a dispenser, a point-of-sale device or terminal, or a computing device viewable by the vehicle operator. In some embodiments, providing the graphical communication can include storing the graphical communication in a database. In some embodiments, the graphical communication can be provided on a display screen outside of the dispensing environment, such as on a computing device associated with the vehicle operator and/or a computing device configured with a loyalty program application associated with the dispensing environment.

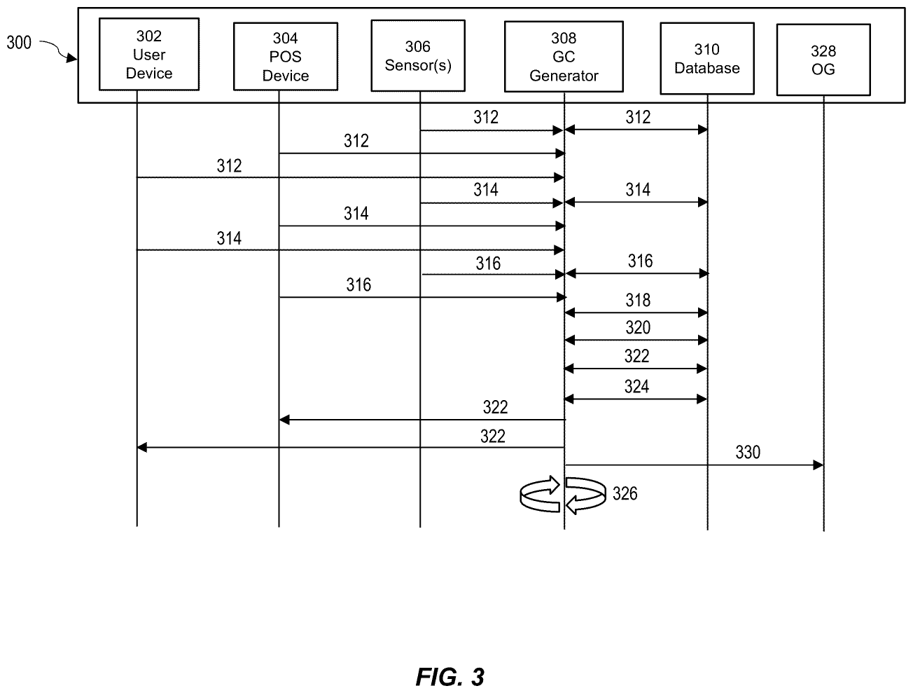

FIG. 3 is a dataflow diagram corresponding to the methods 100 and 200 described in relation to FIGS. 1 and 2. The dataflow of FIG. 3 can be performed by the system 300, which can include a user device 302, a point-of-sale (POS) device 304, one or more sensors 306, a graphical communication (GC) generator 308, a database 310, and an olfactory generator 328 configured to generate sensory reinforcements such as a smell, aroma, odor, fragrance or the like. The system 300 can be configured to adaptively determine, generate, and provide sensory reinforcements and graphical communications associated with promotions offered within a dispensing environment.

First data 312 characterizing a vehicle present within a dispensing environment can be provided to and/or received at the GC generator 308 from the user device 302, the POS device 304, the one or more sensors 306 and/or the database 310. Additionally, second data 314 characterizing an operator of the vehicle can be provided to and/or received from user device 302, the POS device 304, the one or more sensors 306 and/or the database 310. Additionally, or alternatively, the GC generator 308 can receive additional input data 316 from the POS device 304, sensors 306, and/or the database 310. The GC generator 308 can determine one or more vehicle attributes 318 based on the first data 312 and an identity 320 of the vehicle operator based on the second data 314. In some embodiments, the one or more vehicle attributes 318 and the identity 320 of the vehicle operator can be provided to and/or received from the database 310.

Responsive to determining the one or more vehicle attributes 318 and the identity 320 of the vehicle operator, the GC generator 308 can determine third data 322 including a graphical communication including graphical elements providing textual, graphical, and/or computer-executable content related to a promotion associated with the dispensing environment. In some embodiments, the third data 322 can be provided to and/or received from the database 310. The GC generator 308 can determine fourth data 324 characterizing a likelihood the operator will provide an input selecting at least one graphical element in the graphical communication. In some embodiments, the additional input data 316 can be further used to determine the fourth data 324. Based on the fourth data 324, the GC generator 308 can determine the third data 322. In some embodiments, the fourth data 324 can be provided to and/or received from the database 310. The determined third data 322 can be provided to the POS device 304 and/or the user device 302 for viewing by the operator of the vehicle.

In some embodiments, the third data 322 can be used to determine a sensory reinforcement, such as an aroma, a scent, or a fragrance, that can be emitted by the olfactory generator (OG) 328 within the dispensing environment responsive to control signals 330 generated by the GC generator 308. For example, responsive to determining the third data 322, the GC generator 308 can determine, generate, and transmit control signals 330 to a scent generator/emitted that can be configured as the OG 328 within the dispensing environment. The OG 328 can then generate an aroma, scent, or smell associated with graphical communication included in the third data. In some embodiments, the OG 328 can generate the sensory reinforcements in the absence of the third data. The GC generator 308 can iteratively 326 perform one or more aspects of the dataflow based on new or updated first data 312 and/or second data 314 received from any of the user device 302, the POS device 304, sensors 306, and/or the database 310.

FIG. 4 is a system diagram illustrating an example system 500 that incorporates some implementations of the current subject matter and that can provide for the same for adaptively determining and providing graphical communications within a dispensing environment. For example, the system 500 can correspond to the system 300 described in relation to FIG. 3. As shown in FIG. 4, some components of the system 500 can be configured in a dispensing environment 502, such as a retail location at which fuel is sold along with other products and services. Within the dispensing environment 502, one or more computing devices 504 and a forecourt 506 can be provided. The computing devices 504 can be associated with a retail operation, such as a convenience store at which food products and automotive services can be sold. For example, the computing device 504 can correspond to the POS device 304 described in relation to FIG. 3. In some embodiments, the computing device 504 may not be a POS device but can be a computing device arranged within the dispensing environment at which data associated with a vehicle or operator of the vehicle can be acquired.

In some embodiments, the computing device 504 can be a POS device associated with a carwash 508 configured within the dispensing environment 502. The car wash 508 can include one or more sensors 510 that can exchange data with the computing device 504. For example, the sensors 510 can convey car wash availability data and/or car wash utilization data to the computing device 504. For example, the sensors 510 can include image sensors, flow rate sensors, or the like associated with operation and utilization of the car wash 508. In some embodiments, the sensors 510 can generate image data used to determine one or more vehicle attributes and/or image data identifying an operator of a vehicle.

The computing device 504 can include a memory 512 storing computer-executable instructions, which when executed by a processor 514, cause the processor to execute one or more applications associated with operation and control of the car wash, as well as car wash transactions. The computing device 504 can also include a display 516 configured to provide graphical communications as described herein. The computing device 504 can also include one or more input devices 518 such as a microphone, optical scanner, keypad, or the like at which a vehicle operator can provide inputs or data associated with the vehicle or their identity. In some embodiments, inputs can be provided by a user in regard to a graphical communication via the display 516 and/or the input devices 518.

The dispensing environment 502 can also include a forecourt 506. The forecourt 506 can include an area of the dispensing environment in which fuel bays are located for dispensing fuel via one or more dispensers 520. In some embodiments, the dispensing environment 502 can include dispensers configured to dispense other types of “fuel” besides petroleum fuel. For example, the dispensers 520 can be configured to dispense electricity, gas (e.g., hydrogen, liquid propane gas (LPG) or compressed natural gas (CNG), water, or the like. It will be understood that the dispensing environment 502 and the dispensers 520 described herein are not limited to petroleum gasoline in liquid format and that other types of dispensers configured to dispense alternate types of “fuel” can be envisioned.

The dispenser 520 can include a computing device 522. The computing device 522 can include at least one data processor 524 configured to perform one or more aspects of the functionality described herein, and memory 526 for storing computer-executable instructions for performing one or more aspects of the functionality described herein. The computing device 520 can also include a display 528 that is configured to present graphical communications to the dispenser user. The computing device 520 can also include one or more input devices 530 such as a microphone, optical scanner, keypad, or the like at which a vehicle operator can provide inputs and/or data associated with dispensing fuel. In some embodiments, inputs can be provided by a user via the display 528 and/or the input devices 530. In some embodiments, the computing device 522 can be configured to receive control signals generated by the computing device 546 (e.g., from the GC 308 configured on the computing device 546) to cause the dispenser 520 to generate sensory reinforcements, such as an aroma, scent, fragrance, or smell that may or may not be associated with a graphical communication as described herein.

In some implementations, as shown in FIG. 4, the system 500 can include a computing device 532 associated with, corresponding to, or in possession of a vehicle operator. As shown, the computing device 532 can be located within the forecourt 506, such as when the vehicle operator is dispensing fuel via the dispenser 520. In other embodiments, the computing device 532 may not be located within the forecourt 506 or the dispensing environment 502. The computing device 532 can include at least one data processor 534 configured to perform one or more aspects of the functionality described herein, and a memory 536 for storing instructions for performing one or more aspects of the functionality described herein. The computing device 532 can also include a screen or display 538 that is configured to present graphical communications and other data related to the dispensing environment 502. The computing device 532 can also include one or more input devices 540 such as a microphone, optical scanner, camera, keypad, or the like at which a vehicle operator can provide inputs and/or data associated with operation of the computing device 532. In some embodiments, inputs can be provided by the user via the display 538 and/or the input devices 540. In some embodiments, the memory 536 may store a loyalty program application associated with the operator of the vehicle. The loyalty program application can include first data characterizing the vehicle and/or second data characterizing the identity of the vehicle operator.

The computing devices 504, 522, and 532 can be communicatively coupled to one another and to a server 546 via a network 544. The network 544 can include, for example, any one or more of a personal area network (PAN), a local area network (LAN), a campus area network (CAN), a metropolitan area network (MAN), a wide area network (WAN), a broadband network (BBN), a virtual private network (VPN), the Internet, or the like. Further, the network 544 can include, but is not limited to, any one or more of the following network topologies, including a bus network, a star network, a ring network, a mesh network, a star-bus network, tree or hierarchical network, or the like.

In some embodiments, the dispenser 520 can be communicatively coupled to the network 544 via a forecourt controller 542. The forecourt controller 542 can be configured to control operations of the dispenser 520. In some embodiments, the dispenser 520 can be communicatively coupled to the network 544 without a forecourt controller 542.

As further shown in FIG. 4, the system 500 can include a server 546 that is configured to perform operations that adaptively determine, generate, and provide graphical communications associated with the dispensing environment 502. In some embodiments, the server 546 (and/or the network 544) can be configured within the dispensing environment 502. The server 546 can include at least one data processor 548 configured to perform one or more aspects of the functionality described herein, and a memory 550 for storing computer-executable instructions for performing one or more aspects of the functionality described herein. In some embodiments, the server 546 can include a database 552 (e.g., corresponding to database 310 of FIG. 3), which can store data associated with the functionality described herein. For example, the database 552 can store first data 312, second data 314, additional input data 316, as well as loyalty program data.

In some embodiments, the database 552 and/or the memory 550 can store mappings identifying sensory reinforcements that correspond to a particular graphical communication. In other embodiments, the database 552 and/or the memory 510 can store one or more attributes associated with a sensory reinforcement. For example, the attributes can identify a product, a service, a time of day, a dispensing environment condition associated with a given sensory reinforcement. In some embodiments, the sensory reinforcement attributes can also include a type, a medium, a strength, a concentration, a composition, or an emission duration of a sensory reinforcement. In some embodiments, a predictive model trained in a machine learning process can be configured to determine a sensory reinforcement as an output based on an input identifying a graphical communication or an attribute of a sensory reinforcement.

As explained in further detail below, the server 546 can include one or more modules 554 or programs of computer-executable instructions performing one or more aspects of the subject matter described herein using the at least one data processor.

In some embodiments, the one or more modules can include a sensory reinforcement module configured to determine a sensory reinforcement (e.g., a smell) based on sensor data associated with the dispensing environment, a pre-determined and configurable calendar or schedule stored in memory 550 or database 552, or via real-time user input. The sensory reinforcement module can generate control signals associated with the determined sensory reinforcement and the control signals can be transmitted to the dispenser 520. Responsive to receiving the control signals, a scent emitter/generator configured in/on the dispenser 520 can generate a scent, smell, fragrance, or aroma, which can be associated (or not) with the graphical communication provided via the display 528 of the dispenser 520.

FIGS. 5A and 5B illustrate one embodiment of a dispenser 600 that can be used in some implementations of the current subject matter. The dispenser 600 can correspond to dispenser 520 described earlier. In some embodiments, the dispenser 600 includes a dispenser body 601 having an electronics compartment 602, a pump compartment 604, and an image sensor 622. The pump compartment 604 houses a pump configured to pump fuel from a fuel tank or other reservoir, as well as one or more meters that can be configured to monitor fuel flow, flow of fuel additives, and/or flow of other components of the fuel. The pump compartment 604 can also include other components to facilitate fuel dispensing and mixing, such as motors and valves, a strainer/filtering system, a vapor recovery system, and the like. The pump compartment 604 is isolated from the electronics compartment 602 within the dispenser 600 to facilitate safety, security, and/or maintenance, as will be appreciated by a person skilled in the art. Fuel is thus not allowed to flow from the pump compartment 604 to the electronics compartment 602 and instead flows from the pump compartment 604 through hose 606 to a nozzle 608 for dispensing. As will be appreciated by a person skilled in the art, the nozzles 608 are each configured to dispense fuel from the dispenser 600 as pumped therefrom by the pump. The dispenser 600 also includes a nozzle receptacle 609 configured to store the nozzle 608 when not in use.

As referenced above, in some implementations, the dispenser 600 can include one or more components configured to send and/or receive data. For example, dispenser 600 can include a wireless transceiver 624 positioned on the electronics compartment 602 or elsewhere on the dispenser 600, such as on the dispenser body 601. The wireless transceiver 624 can be communicably coupled to the wireless communications module 618. In some embodiments, the wireless transceiver 624 can be a Bluetooth wireless transceiver configured to exchange data with a computing device via short-range radio signals. In some embodiments, one or more sensors 626 can be coupled to the dispenser 600. The one or more sensors 626 can correspond to sensors 306 described in relation to FIG. 3. In some embodiments, the one or more sensors 626 can include image sensors configured to acquire image data associated with the vehicle or the operator of the vehicle at the dispenser 600. In some embodiments, the one or more sensors 626 can include an air quality sensor configured to detect the presence of fuel and/or combustion exhaust fumes or vapors. In some embodiments, the one or more sensors 626 can include a fluid sensor configured to detect a fluid leak from within the forecourt 506. In some embodiments, the one or more sensors 626 may not be configured in or on the dispenser 600, but instead can be configured at one or more locations within the forecourt 506 or the dispensing environment 502.

In some implementations, the dispenser 600 can be configured to dispense electricity and can include several components for dispensing the electricity. For example, dispenser 600 can be configured as an electric vehicle charger. In such embodiments, the dispenser 600 can include a charging cable 628 coupled to the dispenser body 601 at one end and configured to deliver electricity to a charging connector 630 coupled to an opposite end. The charging connector 630 can be configured to couple to a charging port of a vehicle (not shown) and to deliver the electricity provided by the dispenser 600, via the charging cable 628, to the vehicle when coupled to the charging port. When not in use, the charging connector 630 can be stored in a charger receptacle 632 formed on the dispenser body 601.

In some embodiments, the dispenser 600 can include an olfactory generator (OG) 634. The OG 634 can correspond to the OG 328 described in relation to FIG. 3. The OG 634 can include one or more scent generators 636, such as a first scent generator 636A configured to generate a first aroma, fragrance, or smell and a second scent generator 636B configured to generate a second aroma, fragrance, or smell that can be different than the first aroma, fragrance, or smell. In some embodiments, the first scent generator 636A and the second scent generator 636B can be configured to generate the same aroma, fragrance, or smell. In some embodiments, the scent generators 636 can be modular components that can be easily replaced within the OG 634 for maintenance, replacement, or to be configured with respect to a particular graphical communication (and thus promotional offer). In this way, a dispensing environment operator can configure the OG 634 to generate different sensory reinforcements that can be associated with different promotional campaigns.

The scent generators 636 can include a scent-generating medium, such as a stick or capsule containing a particular medium or material configured to emit or otherwise provide a vapor, aroma, or emission that will generate an olfactory response when received within a human body. The scent generators 636 can also include a motorized fan configured to distribute the emission into the surrounding ambient environment. In some embodiments, the scent generator 636 can include a heating element and/or a fluid reservoir containing a fluid, which can each be associated with generating a sensory reinforcement such as a smell, aroma, fragrance or the like.

A variety of smells, aromas, and fragrances can be configured in the scent generators 636. In some embodiments, the smells can be associated with a product or service identified in the graphical communications that can be provided for display at the dispenser 600. For example, the scent generators 636 can be configured to generate a coffee smell or an aroma of coffee when the display 620 is displaying a graphical communication associated with a promotional offer related to a coffee product available within the dispensing environment. In this example, the coffee scent can be generated with graphical communications that are displayed in the morning hours when a vehicle operator may be seeking a coffee beverage. In another example, the scent generators can be configured to generate a smell associated with a food product, such as a smell corresponding to a fried chicken or a hamburger, which can be generated at lunchtime with respect to a graphical communication identifying a promotional offer for fried chicken or a hamburger. In this way, the system herein can advantageously generate sensory reinforcements that are likely to influence a patron of the dispensing environment to make a purchase of the product or service identified in the graphical communication that is associated with the generated smell, aroma, or fragrance. As a result, operators of the dispensing environment are more likely to increase sales and revenue, build customer loyalty, and enhance a purchasing experience for customers.

In some embodiments, the scent generators 636 can be configured to generate obscuring scents, aromas, or fragrances which can be configured to mask undesirable conditions or scents which can be present within the dispensing environment. For example, customers are likely to dislike (or even leave) a dispensing environment that has a heavy smell of fuel or vehicle exhaust fumes. The scent generators 636 can be configured to generate scents, aromas, or fragrances that can mask, counter, or otherwise alter or replace undesirable smells in the dispensing environment so that patrons are more likely to make a purchase of fuel or other products or services in the fuel dispensing environment.

In addition, the OG 634 can include at least one indicator mechanism 638 to provide an indication of operation of the scent generators 636. The indicator mechanism 638 can be further configured to provide status indications regarding an operating or maintenance state of the OG 634 and its components. In some embodiments, the indicator mechanism 638 can include a visible element, such as a light-emitting diode (LED), and/or a speaker configured to generate audible messages.

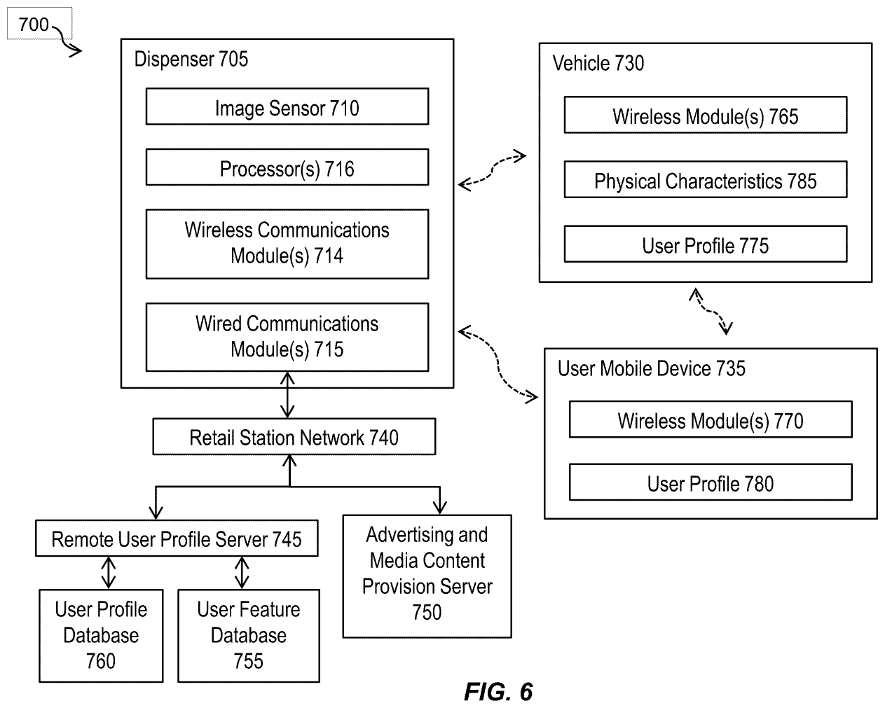

FIG. 6 is a system block diagram illustrating one embodiment of a dispensing system 700. The dispensing system 700 can correspond to the portions of the system 500 shown and described in relation to FIG. 4 and dispensers 705 can correspond to dispensers 520, 600 shown and described in relation to FIGS. 4 and 5A-5B except where noted otherwise. The dispensing system 700 can be configured within a dispensing environment, which can include a plurality of dispensers 705 arranged about a dispensing environment forecourt, a retail sales facility or operation, and/or a vehicle service or maintenance facility or operation. The dispensing system 700 includes a dispenser 705 capable of exchanging data with a dispenser user, a vehicle, and/or a computing device of the dispenser user. The dispenser 705 can perform operations that include, but are not limited to, receiving inputs related to selecting products available via the dispenser 705, performing dispensing transactions, exchanging loyalty program data with users, displaying graphical and textual content associated with goods and services available within the dispensing environment, and receiving user inputs regarding the available goods and services.

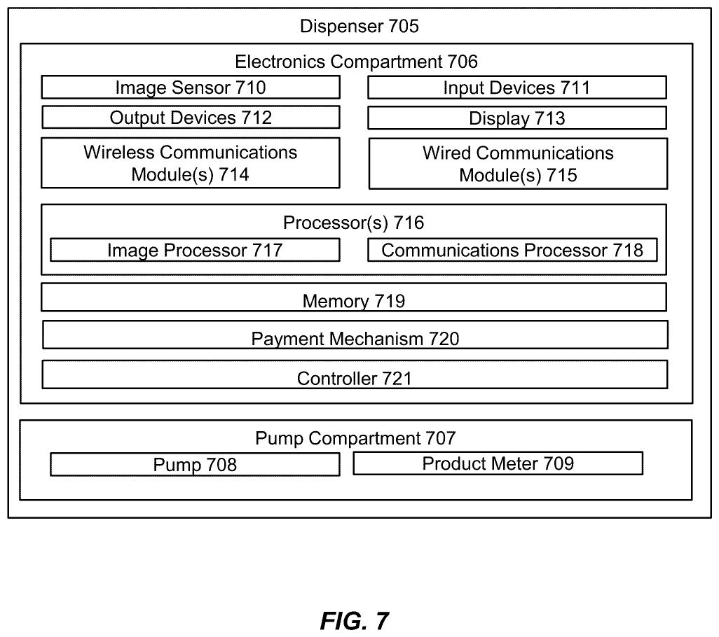

As shown in FIG. 7, the dispenser 705 can include an electronics compartment 706 and a pump compartment 707. The electronics compartment 706 can contain therein electronics for facilitating payment for dispensed products, such as fuel, and for facilitating dispensing of the dispensed products. In some embodiments, the electronics can facilitate payment for goods and services available within the dispensing environment, including but not limited to a food item, a beverage, a parking space, a pharmacy item, groceries to be delivered, a car wash, a tire pressure check, public transit, and the like. The electronics compartment 706 can include an image sensor 710, data processor(s) 716, wireless module(s) 714, wired communications module(s) 715, input devices 711, output devices 712 and a memory 719 or similar non-transitory storage medium configured to store computer-readable and executable instructions, which when executed by the processor 716 perform operations of the dispenser 705 described herein.

The image sensor 710 can include a thermometric camera, an infrared camera, a digital still camera, or a video camera, although other optical sensors are possible. In some embodiments, the image sensor 710 can be affixed to an exterior surface of the dispenser 705. In some embodiments, the image sensor 710 can be configured within the dispensing environment and communicably coupled to the processors 716. The input devices 711 can include an alphanumeric keypad, a numeric keypad, a microphone, or the like. The output devices 712 can include a speaker, a printer, or the like.

The display 713 can be capable of providing information to a user of the dispenser 705. The display 713 can have a variety of configurations, such as a cathode ray tube (CRT) screen, a liquid crystal display (LCD) screen, a light emitting diode (LED) screen, a touchscreen, and the like. For example, the display 713 can include a single display. Alternatively, the display 713 can include multiple displays. For example, a first display 713 can be on a front side of the dispenser 705 and a second display 713 can be on a back side of the dispenser 705. As another example, the display 713 can include two displays mounted next to each other to increase an overall display size. As yet another example, the display 713 can include first and second displays mounted next to each other on a front side of the dispenser 705 and can include third and fourth mounted next to each other on a back side of the dispenser 705.

The communications modules, such as either of the wireless communications module(s) 714 or the wired communications module(s) 715 are capable of exchanging data between the dispenser 705 and computing devices communicably coupled to the dispenser 705. For example, in some embodiments, the wireless communication module(s) 714 can be capable of communicating or exchanging data wirelessly with a remote system (e.g., a remote cloud server, a third-party payment authorization system, etc.) utilizing a variety of communication protocols, e.g., TCP/IP, etc. In some implementations, the wireless communication module(s) 714 can be capable of facilitating wireless communication over a short-range communication link. For example, the wireless communication module(s) 714 can include a transceiver configured to communicate via any of a variety of short-range wireless techniques, such as a Bluetooth protocol, a Wi-Fi protocol, near field communication (NFC), an ultra-wideband (UWB) protocol, a radio frequency identification (RFID) protocol, etc. Any of a variety of types of wireless connectivity hardware can be used for the short-range wireless connectivity, as will be appreciated by a person skilled in the art. The types of wireless connectivity that the wireless communication module(s) 714 includes can be chosen by an owner of the dispensing system 700 according to the owner's current dispensing site setup and/or future dispensing environment plans, and the wireless communication module(s) 714 may be manufactured and/or updated accordingly.

In some embodiments, the wireless module(s) 714 can operatively connect the dispenser 705 with a vehicle 730 and a computing device 735, which in this embodiment is a user mobile device 735, as shown in FIG. 6. The wireless module 714 can include, e.g., a transceiver communicating via Bluetooth protocol, cellular protocol, WIFI protocol, near field communication (NFC), and/or a radio frequency identification (RFID) protocol. The wired communications module 715 operatively connects the dispenser 705 with a remote user profile server 745 and an advertising and media content provision server 750 via a retail station network 740. The retail station network 740 can connect multiple dispensers 705 together over a local area network (LAN).

In some embodiments, the wired communication module(s) 715 can be configured to communicate or exchange data over a wired connection in addition to or instead of over a wireless connection. A wired connection can be used, for example, for a local communication link between the dispenser 705 and a local computing system external to the dispenser 705 (e.g., a forecourt controller, an in-store a point of sale (POS) device, etc.). A wired connection may provide more security and/or stability than a wireless connection and/or may allow a legacy dispenser 705 configured to communicate only via one or more wired connections to implement dynamic management of content provided via the display 713. Wired communication can occur via any of a variety of wired communication protocols, e.g., TCP/IP, etc., as will be appreciated by a person skilled in the art. Some dispensers 705 are manufactured with two-wire connectivity, and the wired communication can accordingly be via two wires, such as via a controller area network bus (CAN Bus) two wire connection, an RS485 two wire connection, a current loop connection, or other type of two wire connection. Some dispensers 705 are additionally or alternatively manufactured with cable connectivity and can accordingly be configured to provide wired communication via cable connection, such as an Ethernet cable or other network cable. Older dispensers 705 typically have two-wire connectivity capabilities while newer dispensers 705 typically have Ethernet connectivity capabilities instead.

The processor(s) 716 can include one or more processors forming part of at least one computing system. In one embodiment, the processor(s) 716 include at least an image processor 717 and a communications processor 718 as shown in FIG. 7. An image processor can receive one or more images from the image sensor 710 and determine identity information of a customer using the images. Identity information can include, for example, facial feature of a customer, a vehicle feature, a license plate number, a non-facial body feature, and the like. The image processor 717 can receive an image from image sensor 710, for example, when the dispenser 705 detects that a customer or user is proximate to the dispenser 705 and/or is in the field of view of the image sensor 710. The image can be of the customer (e.g., can contain a visual representation of the customer) and/or the customer's vehicle, for example. The image processor 717 is capable of performing operations, including but not limited to, receiving image data, and identifying physical characteristics of the user or a vehicle to determine regions within the image data in which the customer's face, body, and vehicle reside.

Using these regions, one or more image features related to the customer's face, body, and vehicle. For example, a facial feature can include skin texture; relative position, size, and/or shape of the eyes, nose, cheekbones, and jaw; and the like. Body features can include height, weight, hair color, body shape, and the like. Vehicle features can include shape, color, license plate number, manufacturer/make/model decal, and the like.

In at least some implementations, the image processor 717 is capable of classifying aspects of the image data as a vehicle, a non-facial body part, and/or a safety object or event. For example, the image processor 717 can classify (or determine) characteristics of the customer's vehicle based on the vehicle features. These characteristics can include, for example, license plate number, vehicle make, required grade and/or type of fuel for the vehicle, and vehicle model.

The image processor 717 is also capable of classifying (or determining) characteristics of the customer that do not directly derive the customer's identity based on the non-facial body features. For example, the image processor 717 is capable of determining a customer's height, weight age, gender, disability status (e.g., in a wheelchair or not in a wheelchair, etc.), and the like.

The image processor 717 is further capable of classifying (or determining) behavior of the customer that relates to safety and is based on an extracted feature present within the image data. For example, the image processor 717 can determine whether the customer is smoking, whether the customer is grounded prior to dispensing products or fuel, whether the vehicle engine is running during fueling, and whether the customer is about to “drive-off” (which can include leaving the fuel retailer without paying for dispensed products or fuel). Other determinations can include environmental, mechanical, electrical, and/or logical instruction conditions, such as, for example, temperature, pressure, humidity, fuel leaks, open panels, dispenser intrusion, power irregularities, watchdog timer expiration, and software exceptions.

Based on these classifications, the image processor 717 is capable of generating an alarm. The alarm can include a warning (e.g., signal, audio, light, and the like) to an attendant of the dispensing environment, such as at a site of the dispenser 705. The warning can include an audible sound emanating from the dispenser 705, a visual or graphical warning on the display 713 of the dispenser 705 indicating that products cannot be dispensed until the detected problem is corrected, and the like. Generating the alarm can include causing a corrective action to be performed, for example, restarting the dispenser 705 (e.g., in the event that a mechanical, electrical, and/or logical problem with the dispenser 705 is detected by the image processor 717), shutting down the dispenser 705 (e.g., in the event that an unsafe condition is detected by the image processor 717, such as the customer smoking before or during fueling, the customer not being grounded prior to dispensing fuel or products, the vehicle engine running during fueling, or a mechanical, electrical, and/or logical problem with the dispenser 705 being detected that cannot be fixed without manual intervention), downloading instructions for the dispenser 705 (e.g., to correct a mechanical, electrical, and/or logical problem with the dispenser 705), and/or generating notifications for other components at the fueling facility that includes the dispenser 705 (e.g., in the event an unsafe condition is detected by the image processor 717 that may affect safe functioning one or more other dispensers 705 within the dispensing environment).

In at least some implementations, image data including the facial features of a user or customer can be conveyed via the dispenser's communications module(s), such as the wireless module(s) 714 and/or the wired communications module(s) 715 to a remote user profile server 745, as described more fully below.

Referring again to FIG. 6, the dispenser 705 can transmit image data including facial features of a user or customer to a remote user profile server 745 in order to match the customer with a known customer identity. The remote user profile server 745 can receive the facial features and access a database 755 (which may include one or more databases) containing known customer features. The database 755 can contain features of customers that have previously visited the dispenser 705 or that have previously enrolled in a customer rewards program associated with the facility providing the dispenser 705 and provided an image of their face in association with the program. The database 755 can also associate unique identities (e.g., names or unique identifiers) with known facial features, e.g., in a table. The remote user profile server 745 can compare the received facial features with the features in the database 755 to find a match. If and once a match is found, the remote user profile server 745 can use the associated customer identifier to query a user profile database 760. The user profile database 760 can contain user profiles for each known customer in the feature database 755 (which may include one or more databases). User profiles can include preferences related to dispensed products (e.g., a preferred product grade, a product type, a payment method, a loyalty rewards identifier, whether to apply loyalty rewards to a present purchase, whether to purchase a car wash, and the like). The user profile and/or identity can be transmitted from the remote user profile server 745 to the dispenser 705.

The user profile and/or identity may be received by the communications processor 718 and can be stored in the memory 719. The user profile can be used by the communications processor 718 to provide a customized product dispensing experience. For example, the user profile can be accessed and the dispenser 705 can be configured with the customer's preferences. This can include rendering, on the display 713, a preference selection screen populated with the customer's dispensing preferences as specified in the user profile. In at least some implementations, the dispenser 705 can render a personalized greeting on the display 713.

In at least some implementations, identity information can be received by the communications processor 718. The identity information can include a name or unique identifier of the customer. This identity information can be used by the communications processor 718 to acquire the user profile from the remote user profile server 745. In at least some implementations the identity information can include, for example, facial features of the customer, vehicle features, license plate number, non-facial body features, and the like.

In at least some implementations, the user identity can be provided to an advertising and media content provision server 750, which can provide customized or targeted advertisements and content to the dispenser 705 for provision to the customer during dispensing of products, e.g., by displaying the advertisements and content on the display 713. For example, once the user identity is determined, advertisements can be dynamically determined and provided.

The advertisements can be pre-specified by remote merchants. Remote merchants can be any appropriate sellers of goods and/or services. For example, a merchant may sell durable goods (e.g., vehicle parts, toys, etc.), perishable goods (e.g., food, drink, etc.), intangible goods (e.g., software, digital media, etc.), or services (e.g., oil changes, car washes, etc.). Remote merchants can include any appropriate computer systems (e.g., servers and databases) for allowing them to send data regarding their goods and/or services over a communication network to fuel dispensers. Remote merchants can operate proactively, interactively, and/or or passively with fuel dispensers to market and/or sell their goods and/or services. For example, the remote merchants can download merchandising content (advertisements and pricing data) to the dispensers 705 at designated times or events, or the remote merchants can download merchandising content to the dispensers 705 upon request. In at least some implementations, the remote merchants can maintain a Web-based portal through which the dispensers 705 can download the content. As discussed herein, remote merchants are remote in the sense that they are not located at the dispensing environment that includes the dispensers 705 to which the remote merchants provide advertisements and/or other content. Thus, the remote merchants can be located in the neighborhood of the dispensing environment. One or more the merchants, of course, could be located at greater distances (e.g., across the state or country) from the dispensing environment.

Dynamic advertisements can include a listing of goods and/or services, along with descriptions and pricing information. The advertisements can include text, graphics, audio, and/or video for presentation at the dispenser 705.

Using the user profile and/or user identity information, the dispensers 705 can determine when to present the above-described merchant-provided data. For example, a dispenser 705 may present the data (e.g., on a display thereof) at certain points of a product dispensing session (e.g., while a product or a fuel is being dispensed or after dispensing the product or fuel is complete). The dispenser 705 can then determine whether the customer indicates interest in the merchant data (e.g., by detecting user input regarding the presented data). If the dispenser 705 detects user interest in the merchant-provided data, the dispenser 705 can present additional information regarding the goods and/or services and determine whether the customer desires to order a good and/or service. Additional information regarding goods or services can include textual descriptions, images, audio, and/or video.

If a customer desires to order a good and/or service, the dispenser 705 can acquire order data (e.g., quantity, price, and delivery information) or the order data can be included or inferred from the customer's user profile. The dispenser 705 can also acquire payment data or payment data may be included or inferred from the user profile. The dispenser 705 can also evaluate whether the payment data is sufficiently complete. If the payment data is acceptable, the dispenser 705 can then generate a message for the appropriate remote merchant regarding the order and payment information and generate a receipt for the customer. The appropriate merchant can then make arrangement for delivery of the good and/or service.

To facilitate customer interaction in at least some embodiments, the user profile can include customer-related data. The customer-related data can, for example, be associated with a customer identifier (e.g., a credit card number, a personal identification number (PIN), a telephone number, a radio frequency identifier (RFID) number, or a loyalty program number). The customer-related data can be information regarding a product dispensing session (e.g., a type of product or fuel, a display language for the dispenser display, audio settings for the dispenser, or payment preferences (e.g., certain credit card, certain debit card, cash to be paid at a staffed payment terminal, etc.)), data regarding services at the dispensing environment (e.g., car wash, air pump, or water hose), or data regarding the customer (e.g., address and preferred payment types). In at least some implementations, the customer-related data can be used to identify other information that may be of interest to the customer. For example, particular types of merchandise (e.g., drinks, newspapers, or food) or offers (e.g., coupons or advertising) can be presented to the customer based on customer-related data. This presentation can, for example, be based on the customer's past purchasing habits in a fueling facility store. The customer-related data can be acquired from the user profile and/or from a remote server using the customer identity.

In at least some implementations, the dispenser 705 can receive the user profile directly from a vehicle 730 (e.g., the customer's vehicle) and/or the mobile device 735. Each of the vehicle 730 and the mobile device 735 can include wireless module(s) 765, 770 (respectively) in communication with one another and with the wireless module(s) 714 of the dispenser 705. Communications between the vehicle 730 and the dispenser 705 can use an on-board diagnostics (OBD) mechanism of the vehicle 730, e.g., OBDII technology in which the vehicle 730 includes an OBDII port (cars manufactured after 1996 have an OBDII port). A copy of the user profile 780 can be contained on the customer's vehicle (for example, a smart vehicle having at least one data processor forming part of at least one computing system with the user profile stored in a memory of the at least one computing system) and/or a copy of the user profile 775 can be contained on the customer's mobile device 735 (e.g., in a memory thereof). When the dispenser 705 detects that the customer is proximate thereto (for example, via the image sensor 710 and/or the wireless module(s) 714), the wireless module(s) 714 can initiate a communication session with the vehicle 730 and/or the mobile device 735 and retrieve the customer's user profile. Once the user profile is received directly from the vehicle 730 or the mobile device 735, the customized dispensing experience can be provided as described above.

Returning to FIG. 7, the electronics compartment 706 can also include a payment mechanism 720 (e.g., a card reader, a Near Field Communication (NFC) module, etc.) configured to facilitate payment for dispensed products, such as fuel, (or other goods and services). The payment mechanism 720 can be configured to receive inputs such as, e.g., user identification information and/or payment information, and deliver the information to the controller 721. For example, the payment mechanism 720 can include a barcode and/or QR code scanner, and/or an NFC contactless card reader for receiving payment information, user identification information, vehicle information, and/or loyalty program information.

The electronics compartment 706 can also include a controller 721 configured to receive instructions from the processor(s) 716 and generate one or more control signals controlling operations of components of the dispenser 705 in accordance with the operations described herein. In some embodiments, the controller 721 can include a data processor and a memory storing computer-readable and executable instructions, forming part of at least one computing system within the electronics compartment 706. In some embodiments, controller 721 can be operably coupled to components of the electronics compartment 706, such as the display 713, the image sensor 710, the wireless communication module(s) 714, the wired communication module(s) 715, the processor(s) 716, the memory 719, and the payment mechanism 720, and the controller 721 can be configured to control operations thereof. In some embodiments, the controller 721 can be configured as a fuel controller and can be operatively coupled to components of the pump compartment 707, such as the pump 708 or the product meter 709. The controller 721 can generate control signals controlling operations of the pump 708 or the product meter 709.

The controller 721 can be further configured to receive from and/or control the one or more sensors 622, 626. The controller 721 can be communicatively coupled to the OG 634 and can provide control signals thereto to cause the OG 634 to generate a sensory reinforcement, such as a smell, an aroma, or a fragrance from the scent generators 636. The controller 721 can also generate and provide controls signals associated with any components of the OG 634, such as the indicator mechanism 638.

The pump compartment 707 houses a pump 708 configured to provide a liquid dispensed product, such as fuel, from a storage tank or other reservoir. The pump compartment 707 can also include one or more product meters 709 that can be configured to monitor flow of dispensed products, flow of additives added to the dispensed product, and/or flow of other components of the dispensed product fuel. The pump compartment 707 can also include other components to facilitate product dispensing and mixing, such as motors and valves, a strainer/filtering system, a vapor recovery system, and the like. The pump compartment 707 is isolated from the electronics compartment 706 within the dispenser 705 to facilitate safety, security, and/or maintenance, as will be appreciated by a person skilled in the art. Dispensed products do not flow or are not conveyed from the pump compartment 707 to the electronics compartment 706 and instead the dispensed products, such as fuel, flow or otherwise are conveyed through the pump compartment 707 to a dispensing device of the dispenser 705, such as a hose and a nozzle at an end of the hose. The dispenser 705 can include any number of hoses and associated nozzles.

A person skilled in the art will appreciate that the dispenser 705 can have various other configurations. Various exemplary implementations of dispensers and methods of provisioning software thereto are described further in, for example, U.S. Pat. No. 10,214,411 entitled “Fuel Dispenser Communication” issued Feb. 26, 2019; U.S. Pat. No. 10,269,082 entitled “Intelligent Fuel Dispensers” issued Apr. 23, 2019; U.S. Pat. No. 10,577,237 entitled “Methods And Devices For Fuel Dispenser Electronic Communication” issued Mar. 3, 2020; U.S. Pat. No. 10,726,508 entitled “Intelligent Fuel Dispensers” issued Jul. 28, 2020; U.S. Pat. No. 11,276,051 entitled “Systems And Methods For Convenient And Secure Mobile Transactions” issued Mar. 15, 2022; U.S. Pat. No. 11,429,945 entitled “Outdoor Payment Terminals” issued Aug. 30, 2022; U.S. Pat. No. 11,443,582 entitled “Virtual Payment System and Method for Dispensing Fuel” issued Sep. 13, 2022; U.S. Pat. App. Pub. No. 2023/0196360 entitled “Conducting Fuel Dispensing Transactions” published Jun. 22, 2023, and U.S. Pat. App. Pub. No. 2023/0103400 entitled “Intelligent Electronic Fueling Station Component Provisioning” published Apr. 6, 2023, each of which are hereby incorporated by reference in their entireties.

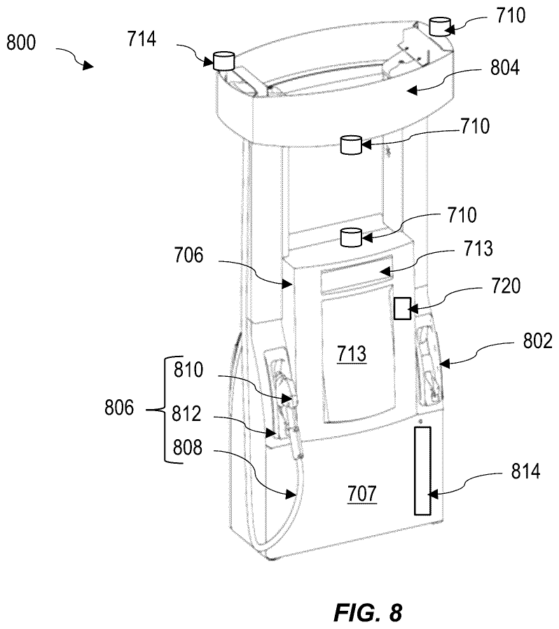

FIG. 8 illustrates a perspective view of one embodiment of a dispenser 800. The dispenser 800 is an embodiment of dispenser 705 of FIGS. 6 and 7. The dispenser 800 can be configured to dispense liquid products (e.g., petroleum fuel). For example, in some embodiments, the dispenser 800 can be configured to dispense liquid products such as gasoline, diesel fuel, ethanol-based fuels, biofuels, diesel exhaust fluid (DEF), fuel additives (e.g., acetone, ether, nitrous oxide, nitromethane, butyl rubber, ferox, oxyhydrogen), water and the like.

As shown in FIG. 8, the dispenser 800 can include a dispenser body 802 in which the electronics compartment 706 and the pump compartment 707 are configured. The dispenser 800 can also include a dispenser awning 804 coupled to the dispenser body 802. In some embodiments, the dispenser body 802 can include the dispenser awning 804. In some embodiments, the dispenser body 802 can exclude the dispenser awning 804. The dispenser awning 804 can include at least one image sensor 710 and at least one wireless transmission module 714 configured thereon. In some embodiments, the dispenser body 802 can, additionally or alternatively, include an image sensor 710. As further shown, the dispenser body 802 can include a display 713, a payment mechanism 720, and a dispensing assembly 806.

The dispenser body 802 can include an electronics compartment 706 and a pump compartment 707. The pump compartment 707 is isolated from the electronics compartment 706 within the dispenser 800 to facilitate safety, security, and/or maintenance, as will be appreciated by a person skilled in the art. Dispensed products or fuel is thus not allowed to flow from the pump compartment 707 to the electronics compartment 706 and instead flows from the pump compartment 707 to the dispensing assembly 806. The dispensing assembly 806 can include a hose 808 coupled to a nozzle 810 for dispensing the liquid product. As will be appreciated by a person skilled in the art, the nozzle 810 can be configured to dispense the liquid product from the dispenser 800 as pumped therefrom by the pump 708. The dispensing assembly 806 can also include a nozzle receptacle 812 configured to store the nozzle 810 when not in use. In some embodiments, the dispenser 800 can include 1, 2, 3, 4, 5, or 6 dispensing assemblies 806. In some embodiments, one or more first dispensing assemblies 806 can be provided on a first side of the dispenser 800 and one or more second dispensing assemblies 806 can be provided on a second side of the dispenser 800 that is opposite the first side of the dispenser 800.

In some embodiments, the dispenser 800 can be configured to dispense diesel exhaust fluid (DEF) and can include a heater 814 within the pump compartment 707 of the dispenser body 802. The heater 814 can be configured to heat the DEF and portions of the pump compartment 707 and/or dispensing assemblies 806. Heating components of the dispenser 800 can be advantageous in climates where freezing temperatures are a concern.

In some implementations, the dispensers described herein can be configured to other types of dispensed products, in addition to or instead of a liquid dispensed product. For example, the dispenser can be configured to dispense products in a gaseous format, such as hydrogen, compressed natural gas (CNG), liquified natural gas (LNG), electricity, or the like. It will be understood that the dispensing environments, dispensing systems, and the dispensers described herein are not limited to dispensing products in liquid format and that the dispensing environments, dispensing systems, and the dispensers described herein can, additionally or alternatively, be configured to dispense products in non-liquid product formats, such as a vapor, a gas, or electricity. For example, in some implementations, the dispenser 800 can be a hydrogen dispenser. As another example, in some implementations, the dispenser 800 can be a compressed natural gas dispenser. As yet another example, in some implementations, the dispenser 800 can be an electrical fuel dispenser configured to dispense electricity.

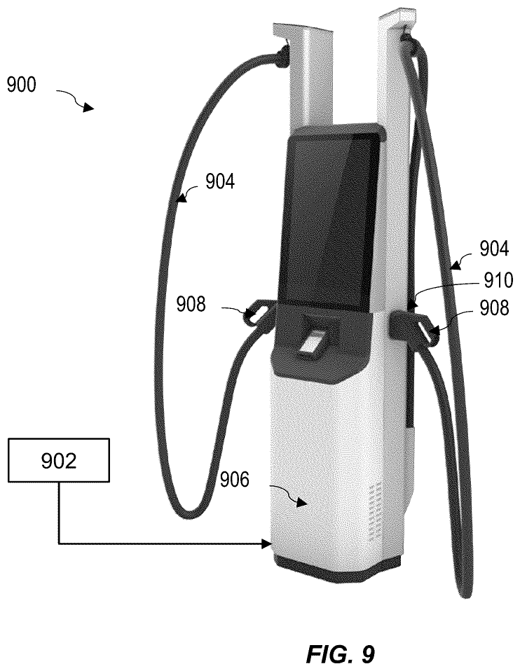

The dispenser 900 of FIG. 9 is another embodiment of the dispenser 705 and 800 of FIGS. 6-8 except where noted otherwise. The dispenser 900 can be configured to dispense electricity. For example, the dispenser 900 can be configured as an electric vehicle charger. The dispenser 900 can be operatively coupled to a power supply 902, such as a local or regional power grid, a battery-back up power supply, a retail sales facility, or a vehicle service facility located in proximity of the dispenser 900.

The dispenser 900 can include a charging cable 904 coupled to a dispenser body 906 of the dispenser 900. In some embodiments, the dispenser 900 can include multiple charging cables 904 as shown in FIG. 9 and is not limited to a configuration having a single charging cable 904. The charging cable 904 can be configured to deliver electricity to a charging connector 908. The charging connector 908 can be configured to couple to a charging port of a vehicle and to deliver the electricity provided by the dispenser 900, via the charging cable 904, to the vehicle when the charging connector 908 is coupled to the vehicle charging port. When not in use, the charging connector 908 is configured to be stored in a charger receptacle 910 formed on the dispenser body 906.

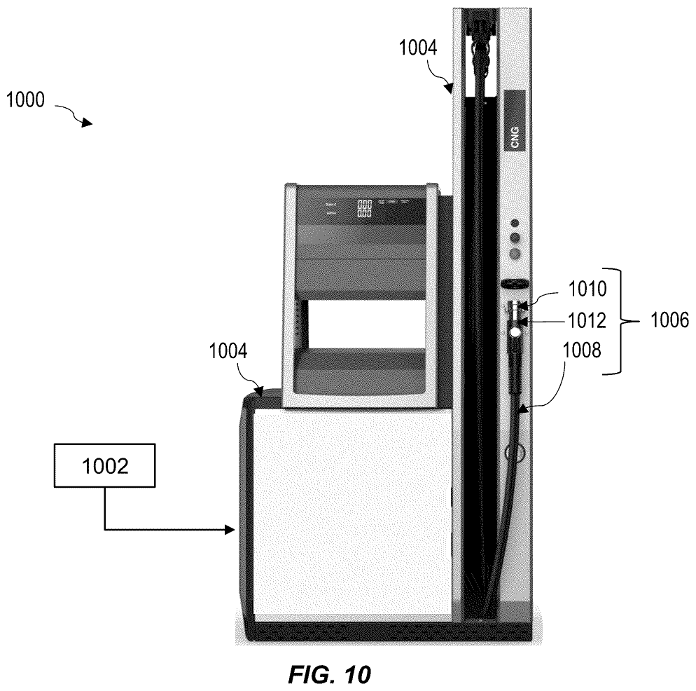

The dispenser 1000 shown in FIG. 10 is another embodiment of the dispenser 705 and 800 of FIGS. 6-8 except where noted otherwise. The dispenser 1000 can be configured to dispense gaseous products such as compressed natural gas (CNG). In some embodiments, the dispenser 1000 can alternatively be configured to dispense, liquified petroleum gas (LPG), hydrogen, and liquified natural gas (LNG). For example, the dispenser 1000 can be operatively coupled to a gas supply 1002 of CNG or other gaseous product, such as a local or regional pipeline, a stored gas supply located within the dispensing environment with the dispenser 1000, or a mobile tube trailer in proximity of the dispenser 1000.

The dispenser 1000 can also include one or more dispensing assemblies 1006 configured within the dispenser body 1004. The dispensing assembly 1006 can include a hose 1008 coupled to a nozzle 1010 for dispensing the gaseous CNG product. As will be appreciated by a person skilled in the art, the nozzle 1010 can be configured to dispense the CNG product from the dispenser 1000. The dispensing assembly 1006 can also include a nozzle receptacle 1012 configured to store the nozzle 1010 when not in use. In some embodiments, the dispenser 1000 can include 1, 2, 3, 4, 5, or 6 dispensing assemblies 1006. In some embodiments, one or more first dispensing assemblies 1006 can be provided on a first side of the dispenser 1000 and one or more second dispensing assemblies 1006 can be provided on a second side of the dispenser 1000 that is opposite the first side of the dispenser 1000.

In some embodiments, the dispensers described herein can be configured to dispense multiple product types. For example, a first portion of a dispenser including a first dispensing assembly can be configured to dispense a liquid product, such as petroleum or DEF, and a second portion of the same dispenser can include a second dispensing assembly configured to dispense a non-liquid product, such as electricity or a gaseous product, such as CNG, LNG, LPG, or Hydrogen. A variety of combinations of dispensing portions and assemblies necessary to dispense multiple, different dispensed products can be envisioned within a single dispenser body of a dispenser as described herein.

Exemplary technical effects of the methods, systems, apparatuses, and non-transitory machine-readable storage mediums described herein include, by way of non-limiting example, adaptively determining, generating, and providing a sensory reinforcement associated with graphical communications within a dispensing environment. This system and method can enable more effective generation and provision of graphical communications of promotional offers or advertisements associated with a product or service offered within the dispensing environment by generating and providing smells, aromas, and/or fragrances that can be associated with the products or services and provided with the graphical communication. As a result, increased sales and revenue can be achieved while optimizing the utilization of services and product offerings, such as food or fuel dispensing. Customer loyalty and brand recognition can be enhanced by providing curated graphical communications coincident with generation of sensory reinforcements, such as aroma, smells, or fragrances and vehicle operators can experience increased satisfaction about purchases and/or utilization of the dispensing environment as a result.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a standalone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

What is claimed is:

1. A method comprising:
receiving first data characterizing a state of a dispensing environment;
receiving second data characterizing a graphical communication to be provided on a display screen within the dispensing environment;
determining a sensory reinforcement to be provided in the dispensing environment based on the first data and/or the second data;
generating the sensory reinforcement; and
providing the sensory reinforcement and/or the graphical communication within the dispensing environment.

2. The method of claim 1, wherein the state of the dispensing environment includes at least one of a presence of at least one patron within the dispensing environment, a time of day, a local ambient condition of the dispensing environment is located, a flow of vehicle traffic within the dispensing environment, or a utilization of equipment or services within the dispensing environment.

3. The method of claim 1, wherein, the sensory reinforcement includes at least one of an aroma, a smell, or a fragrance.

4. The method of claim 1, wherein the sensory reinforcement is generated on a predetermined schedule.

5. The method of claim 1, wherein the sensory reinforcement is provided concurrently with the graphical communication.

6. The method of claim 1, wherein the sensory reinforcement is provided non-concurrently with the graphical communication.

7. The method of claim 6, wherein the sensory reinforcement is a mitigating scent configured to remedy undesirable odors present within the dispensing environment.

8. The method of claim 1, wherein the second data characterizing the graphical communication is determined based on at least one attribute of a vehicle present within the dispensing environment and an identity of an operator of the vehicle.

9. The method of claim 1, wherein determining the sensory reinforcement further comprises determining at least one attribute of the sensory reinforcement, the at least one attribute including a type, a medium, a strength, a concentration, a composition, or an emission duration of the sensory reinforcement.

10. The method of claim 9, wherein the sensory reinforcement is determined based on a predictive model trained in a machine learning process to output a predicted sensory reinforcement based on the graphical communication or the at least one attribute of the sensory reinforcement.

11. A dispenser comprising:
a display;
an olfactory generator;
at least one sensor;
at least one data processor; and
a memory storing instructions, which when executed, cause the at least one data processor to perform operations comprising:
receiving first data characterizing a state of a dispensing environment including the dispenser, the state based on sensor data received from the at least one sensor;
receiving second data characterizing a graphical communication to be provided on the display of the dispenser;
determining a sensory reinforcement to be provided via the olfactory generator of the dispenser based on the first data and/or the second data;
generating, by the olfactory generator of the dispenser, a sensory reinforcement; and
providing the sensory reinforcement via the olfactory generator of the dispenser and/or providing the graphical communication via the display of the dispenser.

12. The dispenser of claim 11, wherein the state of the dispensing environment includes at least one of a presence of at least one patron within the dispensing environment, a time of day, a local ambient condition of the dispensing environment is located, a flow of vehicle traffic within the dispensing environment, or a utilization of equipment or services within the dispensing environment.

13. The dispenser of claim 11, wherein, the sensory reinforcement includes at least one of an aroma, a smell, or a fragrance.

14. The dispenser of claim 11, wherein the sensory reinforcement is generated on a predetermined schedule.

15. The dispenser of claim 11, wherein the sensory reinforcement is provided concurrently with the graphical communication.

16. The dispenser of claim 11, wherein the sensory reinforcement is provided non-concurrently with the graphical communication.

17. The dispenser of claim 16, wherein the sensory reinforcement is a mitigating scent configured to remedy undesirable odors present within the dispensing environment.

18. The dispenser of claim 11, wherein the second data characterizing the graphical communication is determined based on at least one attribute of a vehicle present within the dispensing environment and an identity of an operator of the vehicle.

19. The dispenser of claim 11, wherein determining the sensory reinforcement further comprises determining at least one attribute of the sensory reinforcement, the at least one attribute including a type, a medium, a strength, a concentration, a composition, or an emission duration of the sensory reinforcement.

20. The dispenser of claim 19, wherein the sensory reinforcement is determined based on a predictive model trained in a machine learning process to output a predicted sensory reinforcement based on the graphical communication or the at least one attribute of the sensory reinforcement.
